(19) United States
(12) Patent Application Publication
(10) Pub. No.: US 2024/0156956 A1
(43) Pub. Date: May 16, 2024

(54) Adjuvant Composition

(71) Applicant: MEDRx Co., Ltd., Kagawa (JP)

(72) Inventors: Tatsuhiro Ishida, Tokushima-shi, Tokushima (JP); Takashi Nakae, Higashikagawa-shi, Kagawa (JP); Yasushi Miwa, Higashikagawa-shi, Kagawa (JP); Hidetoshi Hamamoto, Higashikagawa-shi, Kagawa (JP)

(73) Assignee: MEDRx Co., Ltd., Kagawa (JP)

(21) Appl. No.: 18/283,447

(22) PCT Filed: Mar. 23, 2022
(86) PCT No.: PCT/JP2022/013631

§ 371 Date: Sep. 21, 2023

(30) Foreign Application Priority Data
Mar. 24, 2021 (JP) 2021-050087

(51) Int. Cl. A61K 39/39; A61K 39/00; A61P 37/04

(52) U.S. Cl. CPC A61K 39/39; A61K 39/0011; A61P 37/04; A61K 2039/55511

(57) ABSTRACT

The present invention provides an adjuvant composition which is excellent in safety and usability as well as can effectively enhance the immunogenicity of an antigen, and a vaccine composition using the adjuvant composition.

### TECHNICAL FIELD

The present invention relates to an adjuvant composition, specifically an adjuvant composition which is excellent in safety and usability as well as can enhance the immunogenicity of an antigen. In addition, the present invention relates to a vaccine composition using the adjuvant composition.

### BACKGROUD ART

An adjuvant is a substance that is administered together an antigen and may enhance an immunological response to the antigen administered. An adjuvant may act in various ways depending on its kind, e.g., (1) it absorbs an antigen to enhance the intake of the adjuvant into antigen-presenting cells; (2) it retains an antigen in a local area for a long period of time to release the antigen gradually so as to prolong antigenic stimulation; or (3) it directly activates immunocompetent cells. Thus, an adjuvant is very advantageous in reducing the dose and the administration number of a vaccine or the amount of an antigen contained in a vaccine.

Therefore, various studies on adjuvants for enhancing the effect of a vaccine have been done. However, adjuvants that have actually been used in a clinical environment. The typical adjuvant is aluminum salts such as aluminum chloride, aluminum hydroxide and aluminum phosphate (hereinafter referred to as “Alum adjuvant”) that have been used in various vaccines (Patent Document 1). Alum adjuvant is not sufficiently effective as an adjuvant and is hardly be mixed uniformly with an antigen due to its insolubility. Alum adjuvant is not necessarily an ideal adjuvant in terms of effect and usability. As adjuvants other than Alum adjuvant, there are adjuvants using squalene or MPL (Monophosphoryl Lipid). However, the adjuvants have any disadvantages of strong side effects and poor solubility in water, although they have a strong adjuvant activity. Thus, it has been desired in clinical environment to develop an adjuvant that can include an enhanced immunological reaction in human body, produce few side effects and improve the usability.

The current development of adjuvants basically involves in modifying an already-used adjuvant to improve the defect of the adjuvant. For example, Patent Document 1 describes an adjuvant composition using Alum adjuvant that have been used in various vaccines.

However, it has not been reported that a composition comprising an organic acid and meglumine or trometamol enhanced the immunogenicity of an antigen.

### PRIOR ART DOCUMENTS

### Patent Documents

Patent Document 1: WO 2016/194685

### SUMMARY OF INVENTION

### Problem to be Solved by the Invention

An object of the present invention is to provide an adjuvant composition which is excellent in safety and usability as well as can enhance the immunogenicity of an antigen, and a vaccine composition using the adjuvant composition.

### Means for Solving the Problem

The present inventors have intensively studied a

vaccine composition for enhancing the skin permeability of an antigen to produce sufficient antibody production ability and have surprisingly found that a mixture of an organic acid and an organic amine, particularly meglumine or trometamol effectively enhances the immunogenicity of an antigen. The present inventors have then prepared a composition comprising lactic acid or malic acid as an organic acid and meglumine or trometamol to confirm the antibody production ability of an antigen by the composition and have found that the composition functions as an adjuvant. In addition, the present inventors have found that an ionic liquid prepared from lactic acid or malic acid and meglumine or trometamol enhances the immunogenicity of an antigen. Based upon the new findings, the present invention has been completed.

Specifically, the present invention provides the following embodiments.
[Item 1] An adjuvant composition comprising an organic acid and meglumine or trometamol.
[Item 2] The adjuvant composition according to the item 1, wherein the organic acid is lactic acid or malic acid.
[Item 3] The adjuvant composition according to the item 1 or 2, which comprises an ionic liquid prepared from an organic acid and meglumine or trometamol.
[Item 4] The adjuvant composition according to the item 3, wherein the ionic liquid is meglumine salt of lactic acid, meglumine salt of malic acid, trometamol salt of lactic acid, trometamol salt of malic acid or a mixture thereof.
[Item 5] A vaccine composition comprising an antigen and the adjuvant composition according to any of the items 1 to 4.
[Item 6] The vaccine composition according to the item 5, wherein the antigen is cancer antigen peptide.
[Item 7] The vaccine composition according to the item 5 or 6 for use in subcutaneous administration.
[Item 8] The vaccine composition according to the item 7, wherein the number of the administration is 2 times.

### Effects of the Invention

The present invention is excellent in safety and usability as well as can effectively enhance the immunogenicity of an antigen. Thus, the present invention enables more effective prophylaxis or treatment of infection such as virus infection and cancer.

### BRIEF DESCRIPTION OF THE DRAWINGS

FIG. 1 shows the antibody titers of anti-OVA-IgG in the serum of BALB/c mice after the administration of each vaccine composition comprising ovalbumin (OVA) as an antigen (Formulation Examples 1 to 5) (first immunization and second immunization) and the antibody titer of anti-OVA-IgG in the serum of untreated BALB/c mice (Comparative Example).

FIG. 2 shows the antibody titer of anti-OVA-IgG in the diluted serum after the second immunization with each vaccine composition comprising ovalbumin (OVA) as an antigen (Formulation Examples 1 to 5).

### DETAILED DESCRIPTION OF THE PREFERRED EMBODIMENTS

As used herein, the term “adjuvant composition” means a composition comprising an organic acid and meglumine or trometamol with the effect of enhancing an immunological response to an antigen. The form thereof is not particularly limited. The adjuvant composition of the present invention may be a mixture of an organic acid and meglumine or trometamol or form an ionic liquid of an organic acid and meglumine or trometamol in the composition.

As used herein, the term “organic acid” means a generic term for acidic organic compounds. Carbon chain(s) in the organic acid may be straight or branched and saturated or unsaturated. The organic acid of the present invention preferably includes a carboxylic acid, but the organic acid may be an organic acid with carboxyl group(s) and an organic acid with hydroxy group(s) in addition to carboxyl group(s). Examples of the organic acid include an organic acid having 3 to 7 carbon atoms. The organic acid is preferably lactic acid and malic acid.

As used herein, the term “ionic liquid” means a Brønsted salt prepared from an organic acid (organic anion) and meglumine or trometamol (organic cation (organic amine)), which is in a viscous liquid form at ordinary temperature and is a melting point of 100° C. or less. The ionic liquid of the present invention may be prepared, for example, by mixing an organic acid and meglumine or trometamol in equimolar amounts or excess amounts at room temperature or with heating. The excess amounts thereof are preferably within 10-time molar amounts.

The ionic liquid of the present invention may be used in combination with an ionic liquid prepared from a different organic acid and a different organic amine. Also, the ionic liquid may comprise 1 to 10 times the amount of water relative to the amount of the ionic liquid.

As used herein, the “organic cation” is a cationic organic compound, and examples thereof include an organic amine, an organic quaternary ammonium cation, a basic amino acid, and an amino sugar.

Examples of the organic cation include an organic amine having 4 to 12 carbon atoms. The organic cation is preferably diethanolamine, triethanolamine, arginine, lysine, asparagine, meglumine, and trometamol. The organic cation of the present invention is preferably meglumine or trometamol.

As use herein, the term “vaccine composition” means a composition comprising an antigen and an adjuvant composition. The form thereof is not particularly limited. The vaccine composition of the present invention can be used as a vaccine for a disease such as cancer and infection.

As used herein, the term “antigen” means a substance that trigger an immune response in the body. Examples thereof include an antigen protein such as bacteria, fungi, virus and an antigen peptide derived from the antigen protein.

Examples of the antigen protein include virus such as OVA, influenza virus, dengue fever virus and human hepatitis virus, bacteria such as Mycobacterium tuberculosis, Pneumococcus and Staphylococcus, fungi such as Cryptococcus and Aspergillus, and cancer antigen such as WT1, but are not limited thereto.

The sequence of the antigen peptide and the length thereof are not particularly limited. For example, a peptide having 2 to 50 amino acids, a peptide having 2 to 30 amino acids, a peptide with a molecular weight of 5000 or less, or a peptide with a molecular weight of 3000 is used as the antigen peptide. Examples of the antigen peptide include a peptide derived from pathogens such as bacteria, fungi and virus; cancer antigen peptide; a peptide derived from peptide hormone, cytokine, growth factor and receptor protein thereof, but are not limited thereto. The antigen peptide also includes a fragment of an antigen peptide such as OVA peptide, dengue fever virus DEN3-ED3, gag and pol of human hepatitis virus or influenza virus, BAGE, CASP8, CEA, Her2/neu, MAGE-1, MAGE-3, MAGE-A4, MART1, MUC1, NY-ESO-1, p53, PSA, PRAME, TRP1, TRP2, ras, SART-1, IFN-α, IL-6, IL-12, IL-17 and IL-23. The OVA peptide is an immunogenic peptide derived from an albumin from egg white which is an egg allergen.

As used herein, the term “cancer antigen” means a protein which can induce and/or activate cancer-specific cytotoxic T cells (CTLs), recognized by the CTLs from tissue or body fluids or cells of a mammalian organism or from antigen-presenting cells derived from a mammalian organism, and the term “cancer antigen peptide” means an immunogenic peptide derived from the cancer antigen.

The cancer antigen peptide may be bound to transmembrane peptide receptors comprising MHC class I and MHC class II molecules presenting the antigen peptide at the cell surface to T cells of the immune system. The cancer antigen peptide may also be bound to intra or extracellular MHC molecules. The cancer antigen peptide may also be bound to intracellular peptide receptors relating to the heat shock protein (Hsp) family. The cancer antigen peptide may be a peptide having the sequence in which one or two amino acids are added, substituted or deleted in the amino acid sequence. Examples of the cancer antigen peptide include a peptide derived from proteins such as WT1, PR1, GPC3, HER-2, MAGE-A1, MAGE-A2, MAGE-A3, gp100, CEA, hTRT, mTERT, PRAME, PSMA, PSA-1 and MUC-1, but are not limited thereto.

The antigen peptide of the present invention may be in the free form or in any pharmacologically acceptable salt form such as an acid salt (e.g., acetic acid salt, TFA salt, hydrochloric acid salt, sulfuric acid salt, phosphoric acid salt, lactic acid salt, tartaric acid salt, maleic acid salt, fumaric acid salt, oxalic acid salt, hydrobromic acid salt, succinic acid salt, nitric acid salt, malic acid salt, citric acid salt, oleic acid salt, palmitic acid salt, propionic acid salt, formic acid salt, benzoic acid salt, picric acid salt, benzenesulfonic acid salt, dodecyl sulfuric acid salt, methanesulfonic acid salt, p-toluenesulfonic acid salt, glutaric acid salt and salts of each amino acid), a metal salt (e.g., alkali metal salt (e.g., sodium salt, potassium salt), alkali earth metal salt (e.g., calcium salt, magnesium salt) and aluminum salt), an amine salt (e.g., triethylamine salt, benzylamine salt, diethanolamine salt, t-butylamine salt, dicyclohexylamine salt, arginine salt, dimethylammonium salt and ammonium salt).

The antigen peptide of the present invention may be synthesized or generated, isolated, and purified according to any method well known in the art.

The adjuvant composition of the present invention may comprise saline ingredient. The saline ingredient is preferably a solution of sodium chloride and water. Specifically, the saline ingredient of the present invention is 0.90% w/v NaCl solution (about 300 mOsm/L or 9.0 g per L), but is not limited thereto. In the most preferable embodiment, the saline ingredient is Dulbecco's phosphate buffered saline without calcium or magnesium (Cellgro catalog No. 21-CV).

In the preferable embodiment, the saline ingredient may be contained in an amount about 50 vol % to 98 vol % of the adjuvant composition. The among of the saline ingredient may be, for example, a range of 60% to 98%, 70% to 98%, 80% to 98%, 90% to 98%, 50% to 60%, 55% to 75%, 63% to 91%, or may be 55%, 60%, 65%, 70%, 75%, 80%, 85%, 86%, 87%, 88%, 89%, 90%, 91%, 91.1%, 91.2%, 91.3%, 91.4%, 91.5%, 91.6%, 91.7%, 91.8%, 91.9%, 92%, 92.1%, 92.2%, 92.3%, 92.4%, 10 92.5%, 92.6%, 92.7%, 92.8%, 92.9%, 93%, 94%, 95%, 96%, 97% or 98%. In the most preferable embodiment, the adjuvant composition of the present invention contains the saline ingredient in an amount about 92 vol %.

The adjuvant composition of the present invention may be prepared by appropriately adjusting a ratio of an organic acid and an organic amine such as meglumine and trometamol. For example, the ratio of an organic acid and meglumine or trometamol may be a range of 1:1 to 10:1, or may be 1:1, 2:1, 3:1, 4:1, 5:1, 6:1, 7:1, 8:1, 9:1 or 10:1.

The vaccine composition of the present invention may comprise commonly-used known additive(s), for example, an antioxidant, a preservative, a thickener. The additives may be used alone, respectively, or two or more of the additives may be combined in appropriate amounts.

Examples of the antioxidant include a water-soluble antioxidant and a hydrophobic antioxidant. Examples thereof include ascorbic acid, sodium hydrogen sulfite, sodium sulfite, erythorbic acid, tocopherol acetate, dibutylhydroxytoluene, tocopherol, sodium pyrosulfite, butylhydroxyanisole and propyl gallate. The amount of the antioxidant in the vaccine composition of the present invention may be appropriately adjusted depending on, for example, the type of the antioxidant. The amount is, for example, 0.01 to 1% by weight relative to the total amount of the composition. Also, the antioxidant may be used alone, or two or more of the antioxidants may be used in combination.

Examples of the preservative include benzoic acid, sodium benzoate, sorbic acid, sodium sorbate, sodium dehydroacetate, parahydroxybenzoic acid, sodium parahydroxybenzoate, ethyl parahydroxybenzoate, propyl parahydroxybenzoate (propylparaben), butyl parahydroxybenzoate, isopropyl parahydroxybenzoate, isobutyl parahydroxybenzoate, propionic acid, sodium propionate, benzalkonium chloride and salicylic acid. Among them, methylparaben, propylparaben, benzalkonium chloride, salicylic acid or a mixture thereof is preferable. The amount of the preservative in the vaccine composition of the present invention may be appropriately adjusted depending on, for example, the type of the preservative. The amount is, for example, 0.01 to 1% by weight relative to the total amount of the composition. Also, the preservative may be used alone, or two or more of the preservatives may be used in combination.

The thickener encompasses an inorganic material and an organic material. Examples of the inorganic material include amorphous silicon dioxide, kaolin (gypsum), diatomite, talc, hydrated silicon dioxide, light anhydrous silicic acid, magnesium silicate, calcium silicate, calcium phosphate and barium sulfate, and examples of the organic material include crystalline cellulose. The amount of the thickener in the vaccine composition of the present invention may be appropriately adjusted depending on, for example, the type of the thickener. The amount is, for example, 1 to 10% by weight relative to the total amount of the composition. Also, the thickener may be used alone, or two or more of the thickeners may be used in combination.

The adjuvant composition of the present invention can exert the effect of enhancing of an immune response by itself. Thus, the adjuvant composition may be administered sequentially and separately from an antigen.

The vaccine composition of the present invention may contain an antigen and the adjuvant composition in the same preparation. In addition, an antigen and the adjuvant composition may be formulated separately, and then mixed and administered at the time of use.

In the present invention, the blending ratio of the antigen and the adjuvant composition may be appropriately determined depending on various factors such as the type of antigen used, the combination of the antigen and the adjuvant composition. In the present invention, the blending ratio is preferably used such that the adjuvant composition has a high concentration.

The adjuvant composition and the vaccine composition of the present invention are suitable for subcutaneous administration and may be applied to the skin. For example, while an antigen is being administered by any method such as the puncture by microneedles, the adjuvant composition may be separately administered by another method such the application to the skin. The adjuvant composition and the vaccine composition of the present invention may be used in the form of a patch.

The adjuvant composition and the vaccine composition of the present invention may be administered to a human and a mammal such as dog, cat, cow, horse and monkey.

The usage and dosage of the adjuvant composition and the vaccine composition of the present invention may be appropriately adjusted depending on various factors such as the type of antigen used, the age and the weight of the subject to be administered. The vaccine composition of the present invention may be administered the second time (booster immunization) 2 to 6 weeks after the first administration (primary immunization) under the same conditions as the first immunization.

### EXAMPLES

Hereinafter, the present invention is specifically illustrated in Examples. The present invention, however, is not intended to be limited thereto by any means.

In the following Examples, substances such as reagent were obtained from the following manufactures. The present invention, however, is not intended to be limited thereto by any means.
Lactic Acid (Japanese Pharmacopoeia): Kenei Pharmaceutical Co., Ltd.
Malic Acid, Trometamol: FUJIFILM Wako Pure Chemical Corporation
Purified Water: Otsuka Pharmaceutical Factory Inc.
Meglumine: Tokyo Chemical Industry Co., Ltd.
Ovalbumin (OVA): Sigma-Aldrich

### Example 1: Preparation of Vaccine Composition

Each ingredient was weighted in the amount (molar ratio) shown in Table 1 below and the ingredients were mixed to prepare each adjuvant composition. The prepared adjuvant composition was then diluted 10-fold with HEPES buffer to obtain a 1 mL solution, and then OVA (100 μg) was dissolved into the solution to prepare each vaccine composition of Formulation Examples 1 to 5.

TABLE 1

| | | | |
|---|---|---|---|
| Formulation Example 1 | Lactic acid (5) | Trometamol (1) | — |
| Formulation Example 2 | Lactic acid (1) | Meglumine (1) | Water (5) |
| Formulation Example 3 | Lactic acid (1) | Trometamol (1) | Water (5) |
| Formulation Example 4 | Malic acid (1) | Meglumine (1) | Water (5) |
| Formulation Example 5 | Malic acid (1) | Trometamol (1) | Water (5) |

### Example 2: Validation of Adjuvant Effect of Each Composition

### Test Method

As experimental animals, BALB/c mice (5-week age, male) (purchased from Japan SLC (Shizuoka, JAPAN)) were used.

BALB/c mice were randomly classified into the administration groups of the vaccine compositions of Formulation Examples 1 to 5 (n=3 per group), and then each vaccine composition of Formulation Examples 1 to 5 (200 μL) was subcutaneously administered to the BALB/c mice in each group. The amount of OVA in the vaccine composition was set at 20 μg per mouse. The time point of the first administration of the vaccine composition was defined as Day 0, and the vaccine composition was additionally administered in the same way on Day 14. The sera on Day 11 (first immunization) and Day 18 (second immunization) were collected from each mouse, the antibody titers in the sera were measured by ELISA method, and the average value thereof was calculated. As Comparative Example, the antibody titer of anti-OVA-IgG in the serum collected from untreated BALB/c mouse was used (n=3).

In addition, the serum after second immunization was diluted 250-fold, 1000-fold, 4000-fold, 16000-fold and 64000-fold with HEPES buffer to prepare each serum diluted 250-fold, 1000-fold, 4000-fold, 16000-fold and 64000-fold, and the antibody titers of anti-OVA-IgG in the sera were measured by ELISA method. A dilution curve was made based on the measured antibody titers to compare and evaluate the antibody titers of Formulation Examples 1 to 5.

### Result

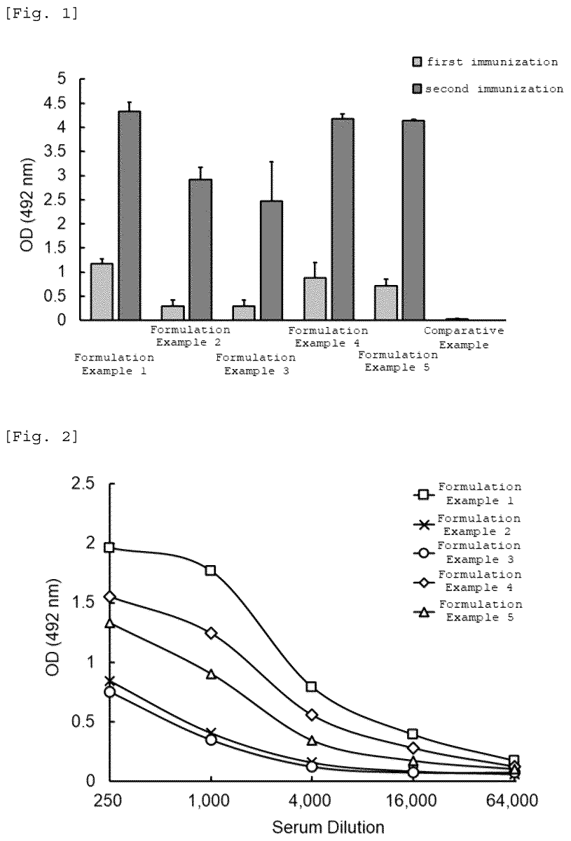

The antibody titers of anti-OVA-IgG in the serum of BALB/c mice after each administration of vaccine compositions of Formulation Examples 1 to 5 (first immunization and second immunization) and the antibody titer of anti-OVA-IgG in the serum of untreated BALB/c mice (Comparative Example) are shown in FIG. 1. The antibody titers of anti-OVA-IgG in the diluted serum of each composition of Formulation Examples 1 to 5 after second immunization is shown in FIG. 2.

FIG. 1 shows that all the compositions of Formulation Examples had high antibody titers of anti-OVA-IgG. The antibody titers after the second immunization were higher as compared to those of first immunization. As a result, it was shown that the immunization with ionic liquids as adjuvant established the acquired immunity system inducing anti-OVA-IgG.

Also, the result on the dilution factor (antibody titer) resulting in OD=0.5 calculated from the dilution curve in FIG. 2 is shown in Table 2. Table 2 shows that the composition of Formulation Example 1 had the highest antibody titer, followed by Formulation Example 4, Formulation Example 5, Formulation Example 2 and Formulation Example 3.

TABLE 2

| | Antibody Titer |
|---|---|
| Formulation Example 1 | 7,126 |
| Formulation Example 2 | 716 |
| Formulation Example 3 | 603 |
| Formulation Example 4 | 4,434 |
| Formulation Example 5 | 2,271 |

The above result shows that the ionic liquids could be used as adjuvant, and they could effectively induce the production of an antibody specifically to an antigen.

### INDUSTRIAL APPLICABILITY

The adjuvant composition of the present invention is excellent in safety and usability as well as can effectively enhance the immunogenicity of an antigen. Thus, the vaccine composition using the adjuvant composition of the present invention enables more effective prophylaxis or treatment of infection such as virus infection and cancer.

1. An adjuvant composition comprising an organic acid and meglumine or trometamol.

2. The adjuvant composition according to claim 1, wherein the organic acid is lactic acid or malic acid.

3. The adjuvant composition according to claim 1, which comprises an ionic liquid prepared from an organic acid and meglumine or trometamol.

4. The adjuvant composition according to claim 3, wherein the ionic liquid is meglumine salt of lactic acid, meglumine salt of malic acid, trometamol salt of lactic acid, trometamol salt of malic acid or a mixture thereof.

5. A vaccine composition comprising an antigen and the adjuvant composition according to claim 1.

6. The vaccine composition according to claim 5, wherein the antigen is cancer antigen peptide.

7. The vaccine composition according to claim 5 for use in subcutaneous administration.

8. The vaccine composition according to claim 7, wherein the number of the administration is 2 times.
